(19) United States
(12) Patent Application Publication
(10) Pub. No.: US 2024/0009075 A1
(43) Pub. Date: Jan. 11, 2024

(54) VERTICAL VIBRATION APPARATUS WITH HEATING FUNCTION

(71) Applicant: HOPE SONG INTERNATIONAL ENTERPRISE CO., LTD., Taipei (TW)

(72) Inventors: Shang-wen SHIH, Taipei (TW); Xiong WANG, Taipei (TW)

(21) Appl. No.: 18/218,134

(22) Filed: Jul. 5, 2023

(30) Foreign Application Priority Data
Jul. 11, 2022 (TW) 111125985

(51) Int. Cl. A61H 23/02; A61F 7/00

(52) U.S. Cl. CPC A61H 23/0254; A61F 7/007; A61F 2007/0086; A61H 2201/0207; A61H 2201/0221; A61H 2201/5082; A61H 2201/0188; A61H 2201/5043; A61H 2201/1669

(57) ABSTRACT

A vertical vibration apparatus with heating function is provided. The vertical vibration apparatus with heating function includes a housing, an actuating device, a driving control unit, and a heating device. The actuating device is disposed in the housing. The driving control unit is disposed in the housing and is electrically connected to the actuating device. The driving control unit is configured to drive and control the actuating device to vibrate. The heating device includes a heating control unit and a heating plate. The heating control unit is disposed in the housing. The heating plate is disposed on a top surface of the housing and is electrically connected to the heating control unit. Therefore, the vertical vibration apparatus with heating function of the present invention can provide a vibration function and a heating function at the same time, so as to achieve a multi-function purpose.

### RELATED APPLICATIONS

This application claims priority to Taiwan Application Serial Number 111125985, filed on Jul. 11, 2022, which is incorporated herein by reference.

### BACKGROUND

### Field of Invention

The present invention relates to vibration apparatus. More particularly, the present invention relates to a vertical vibration apparatus with heating function.

### Description of Related Art

With the advancement in technology and changes in lifestyle, people started to pay attention to physical health and good posture after busy work. Therefore, various kinds of sport equipment or fitness equipment appear on the market, and one of the popular equipment recently is a vibration machine.

The vibration machine helps user's full body or part of the body vibrate, so as to achieve the effect of exercise. However, current vibration machine has only one vibration function and cannot meet the diverse demands. Therefore, it is necessary to provide a multifunctional vibration machine to solve the problem existing in the conventional technique.

### SUMMARY

One object of the present invention is to provide a vertical vibration apparatus with heating function that can provide a heating function while providing a vibration function, so as to provide users with multiple choices and achieve a multi-function purpose.

According to the aforementioned object, a vertical vibration apparatus with heating function is provided. The vertical vibration apparatus includes a housing, an actuating device, a driving control unit, and a heating device. The actuating device is disposed in the housing. The driving control unit is disposed in the housing and electrically connected to the actuating device, and the driving control unit is configured to drive and control the actuating device to vibrate. The heating device includes a heating control unit and a heating plate. The heating control unit is disposed in the housing. The heating plate is disposed on the housing and is electrically connected to the heating control unit.

According to an embodiment of the present invention, the heating control unit comprises a thermostat unit configured to control a temperature of the heating plate.

According to an embodiment of the present invention, the thermostat unit includes a temperature sensor configured to sense a temperature of the heating plate. The heating control unit is configured to be connected to a power supply and is configured to control a working state of the heating plate according to the temperature of the heating plate.

According to an embodiment of the present invention, the temperature sensor is a negative temperature coefficient thermistor.

According to an embodiment of the present invention, the heating plate is a polyimide film heating plate.

According to an embodiment of the present invention, the heating device further comprises a heat conduction plate, and the heat conduction plate covers the heating plate.

According to an embodiment of the present invention, the heating device further comprises an indicator light, and the indicator light is electrically connected to the heating control unit.

According to an embodiment of the present invention, the driving control unit includes a driving unit and a speed stabilization unit. The driving unit is connected to a power supply, and the speed stabilization unit is configured to control a vibration speed of the actuating device.

According to an embodiment of the present invention, the vertical vibration apparatus further comprises an operation display panel electrically connected to the driving control unit and the heating control unit.

According to an embodiment of the present invention, the actuating device comprises a motor, a rotating shaft, an eccentric wheel, a structural body, and a buffer assembly. The eccentric wheel is disposed in the structural body, the rotating shaft passes through the eccentric wheel, and the motor is configured to drive the rotating shaft and the eccentric wheel to rotate, so as to reciprocate the structure body up and down, and the buffer assembly is disposed in the housing.

According to the embodiments of the present invention, in addition to a vertical vibration function, the vertical vibration apparatus of the present invention also has a heating function. In this way, the vertical vibration apparatus facilitates to enhance vibration of user's body, thereby increasing the user's calories consumption and fat reduction, so as to achieve full-body exercise effect. In addition, while exercising, the heating function can help user's blood circulation, thereby reducing user's fatigue and improving exercise performance.

### BRIEF DESCRIPTION OF THE DRAWINGS

The invention can be more fully understood by reading the following detailed description of the embodiment, with reference made to the accompanying drawings as follows:

FIG. 1 is a schematic diagram showing the appearance of a vertical vibration apparatus with heating function according to one embodiment of the present invention.

FIG. 2 is an exploded view of the vertical vibration apparatus with heating function according to one embodiment of the present invention.

FIG. 3 is a partial schematic diagram of the vertical vibration apparatus with heating function according to one embodiment of the present invention.

FIG. 4 is a block diagram illustrating the vertical vibration apparatus with heating function according to one embodiment of the present invention.

### DETAILED DESCRIPTION

In order to make the above and other objects, features, and advantages of the present invention more comprehensible, preferred embodiments of the present invention will be described below in detail together with the attached drawings. Furthermore, the directional terms used in the present invention, for example, up, down, top bottom, front, back, left, right, inside, outside, side, around, central, horizontal, transverse, vertical, longitudinal, axial, radial direction, the uppermost layer, or the lowermost layer, etc. are only the directions shown in the attached drawings. Therefore, the directional terms are only used to illustrate and express the present invention, but not to limit the present invention. %

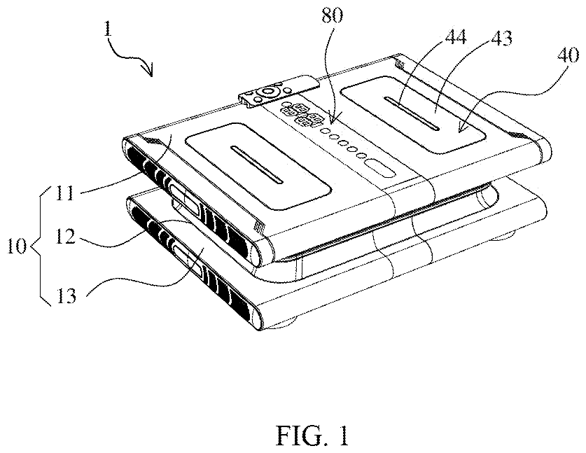

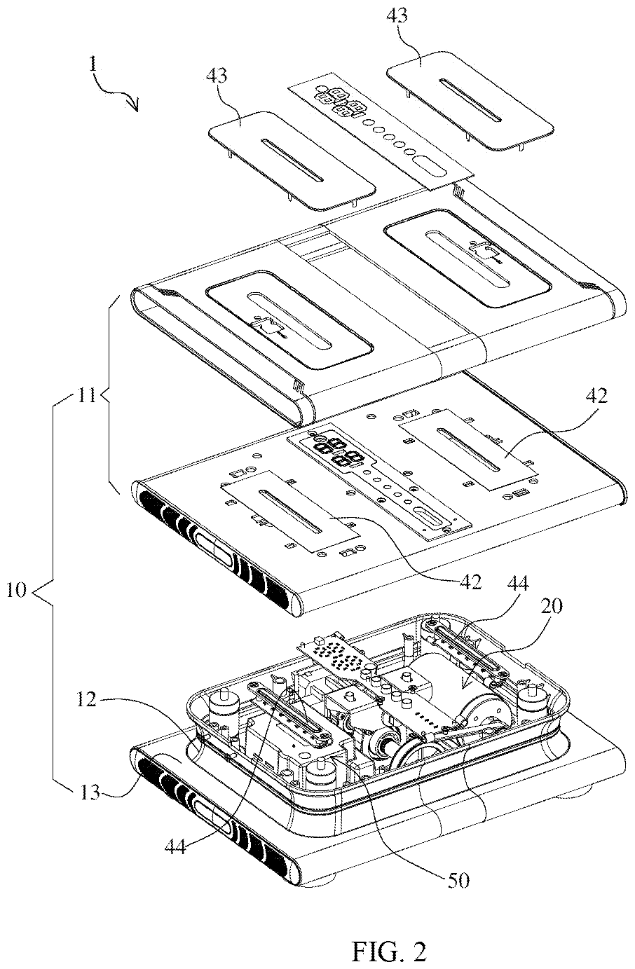

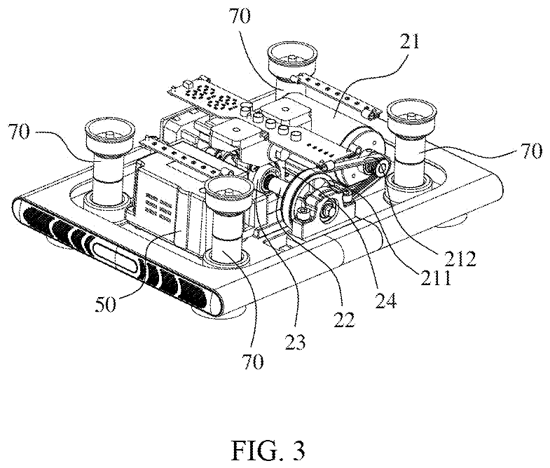

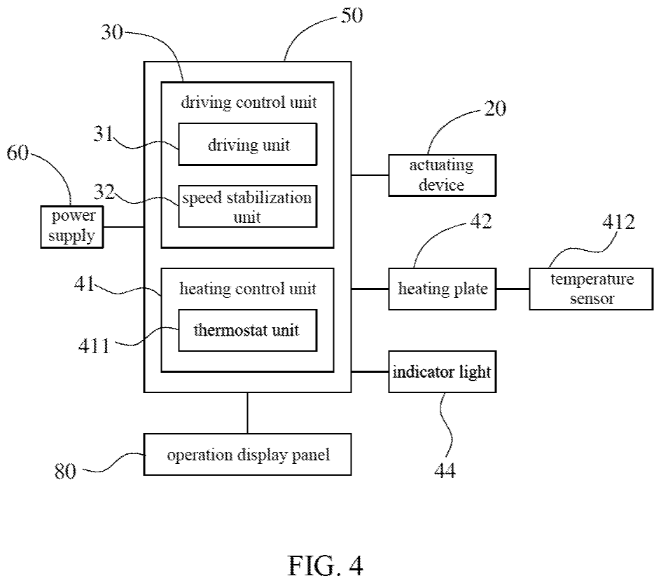

Simultaneously referring to FIG. 1 to FIG. 4, FIG. 1 to FIG. 4 are a schematic diagram, an exploded view, a partial schematic diagram, and a block diagram respectively illustrating vertical vibration apparatus with heating function according to one embodiment of the present invention. The vertical vibration apparatus with heating function 1 in the present embodiment includes a housing 10, an actuating device 20, a driving control unit 30 and a heating device 40. The actuating device 20, the driving control unit 30 and the heating device 40 are disposed in the housing 10. The driving control unit 30 is electrically connected to the actuating device 20 and is configured to control the actuating device 20 to vibrate. The heating device 40 is configured to generate heat on an upper surface of the housing 10.

The housing 10 includes an upper housing 11, a middle housing 12 and a lower housing 13 assembled together. The middle housing 12 is a concave housing. In the present embodiment, the heating device 40 includes a heating control unit 41 and a heating plate 42. The heating control unit 41 is disposed in the housing 10. The heating plate 42 is disposed on an upper surface of the housing 10 and is electrically connected to the heating control unit 41, whereby the heating control unit 41 can control the heating plate 42 to generate heat. In one embodiment, the heating control unit 41 and the driving control unit 30 can be integrated in the form of a circuit board, chip or microcontroller which can be disposed in a control box 50 in the housing 10. The heating control unit 41 and the driving control unit 30 are connected to a power supply 60 to respectively control a working state of the heating plate 42 and a vibration state (such as speed of vibration) of the actuating device 20.

In one embodiment, the heating plate 42 is a polyimide film (PI film) heating plate. The heating device 40 further includes a heat conduction plate 43 and an indicator light 44, and the heat conduction plate 43 covers the heating plate 42. In the present embodiment, the heat conduction plate 43 can be a heat conducting metal plate. The heat conduction plate 43 is configured to contact the user's body and is configured to can uniformize heat distribution generated by the heating plate 42. In one embodiment, the indicator light 44 is electrically connected to the heating control unit 41, and the indicator light 44 is used to display the working state of the heating plate 42. In one example, the indicator light 44 can be, for example, an LED circuit board, and the indicator light 44 can be used to emit different colors of light to indicate different modes of working states. In other embodiments, the heating control unit 41 includes a thermostat unit 411, and the thermostat unit 411 is configured to control the temperature of the heating plate 42. Specifically, the thermostat unit 411 includes a temperature sensor 412, and the temperature sensor 412 is configured to sense the temperature of the heating plate 42, thereby obtaining temperature data in real time. The heating control unit 41 is configured to control the working state of the heating plate 42 according to the temperature of the heating plate 42 sensed by the temperature sensor 412 to maintain the heating plate 42 at constant temperature. For example, the heating control unit 41 controls the heating plate 42 to start heating when the temperature of the heating plate 42 is lower than a predetermined temperature. Moreover, the heating control unit 41 controls the heating plate 42 to stop heating when the temperature of the heating plate 42 is greater than or equal to the predetermined temperature. On the other hand, the heating control unit 41 can also control the heating plate 42 to stop heating when an abnormality occurs, thereby protecting the entire apparatus from overheated. In one example, the temperature sensor is a negative temperature coefficient (Negative Temperature Coefficient, NTC) thermistor.

In the present embodiment, the driving control unit 30 includes a driving unit 31 and a speed stabilization unit 32. The driving unit 31 is connected to the power supply 60, and the speed stabilization unit 32 is configured to control a vibration speed of the actuating device 20. Specifically, the actuating device 20 includes a motor 21, a rotating shaft 22, an eccentric wheel 23 and a structural body 24. The eccentric wheel 23 is disposed in the structural body 24, and the rotating shaft 22 passes through the eccentric wheel 23. The motor 21 is connected to the rotating shaft 22 through a transmission wheel 211 and a transmission belt 212. Thereby, the motor 21 can drive the rotating shaft 22 and the eccentric wheel 23 to rotate via the transmission wheel 211 and the transmission belt 212, so as to reciprocate the structural body 24 up and down. In one embodiment, the vertical vibration apparatus with heating function 1 further includes a buffer assembly 70 disposed in the housing 10. Specifically, the buffer assembly 70 is connected between the upper housing 11 and the lower housing 13, and the buffer assembly 70 provides shock absorption for the actuating device 20 and the housing 10.

In one embodiment, the vertical vibration apparatus with heating function 1 further includes an operation display panel 80. The operation display panel 80 is electrically connected to the driving control unit 30 and the heating control unit 41. Specifically, the operation display panel 80 inclines an operation circuit and a display circuit. A user can set operation modes and parameters of the vertical vibration apparatus through a user interface or a controller for the operation display panel 80, and the operation display panel 80 further display the operation states of vertical vibration apparatus.

According to the embodiments of the present invention, in addition to a vertical vibration function, the vertical vibration apparatus of the present invention also has a heating function. In this way, the vertical vibration apparatus facilitates to enhance vibration of user's body, thereby increasing the user's calories consumption and fat reduction, so as to achieve full-body exercise effect. In addition, while exercising, the heating function can help user's blood circulation, thereby reducing user's fatigue and improving exercise performance.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A vertical vibration apparatus with heating function, comprising:
a housing;
an actuating device disposed in the housing;
a driving control unit disposed in the housing and electrically connected to the actuating device, wherein the driving control unit is configured to drive and control the actuating device to vibrate; and
a heating device comprising:
a heating control unit disposed in the housing; and
a heating plate disposed on the housing and electrically connected to the heating control unit;
wherein the heating control unit comprises a thermostat unit, and the thermostat unit is configured to control a temperature of the heating plate:
wherein the thermostat unit includes a temperature sensor configured to sense a temperature of the heating plate in real time; and wherein the heating control unit is configured to be connected to a power supply and is configured to control a working state of the heating plate according to the temperature of the heating plate sensed by the temperature sensor.

2. The vertical vibration apparatus of claim 1, wherein the temperature sensor is a negative temperature coefficient thermistor.

3. The vertical vibration apparatus of claim 1, wherein the heating plate is a polyimide film heating plate.

4. The vertical vibration apparatus of claim 1, wherein the heating device further comprises a heat conduction plate, and the heat conduction plate covers the heating plate.

5. The vertical vibration apparatus of claim 1, wherein the heating device further comprises an indicator light, and the indicator light is electrically connected to the heating control unit.

6. The vertical vibration apparatus of claim 1, wherein the driving control unit comprises a driving unit and a speed stabilization unit, wherein the driving unit is connected to a power supply, and the speed stabilization unit is configured to control a vibration speed of the actuating device.

7. The vertical vibration apparatus of claim 1, further comprises an operation display panel electrically connected to the driving control unit and the heating control unit.

8. The vertical vibration apparatus of claim 1, wherein the actuating device comprises a motor, a rotating shaft, an eccentric wheel, a structural body, and a buffer assembly, wherein the eccentric wheel is disposed in the structural body, the rotating shaft passes through the eccentric wheel, and the motor is configured to drive the rotating shaft and the eccentric wheel to rotate, so as to reciprocate the structure body up and down, and the buffer assembly is disposed in the housing.
